(19) United States
(12) Patent Application Publication
(10) Pub. No.: US 2006/0272501 A1
(43) Pub. Date: Dec. 7, 2006

(54) Sintered adsorbents, preparation method thereof and use of same for the drying of organic compounds

(76) Inventor: Dominique Plee, Lons (FR)

Correspondence Address: MILLEN, WHITE, ZELANO & BRANIGAN, PC, 2200 CLARENDON BLVD, SUITE 1400, ARLINGTON, VA, 22201, US

(21) Appl. No.: 10/545,169

(22) PCT Filed: Feb. 10, 2004
(86) PCT No.: PCT/FR04/00298

§ 371 Date: May 11, 2006

(30) Foreign Application Priority Data
Feb. 11, 2003 (FR) 03/01596

(51) Int. Cl. B01D 53/02; B01D 59/26; C02F 1/28

(52) U.S. Cl. 95/141; 210/690; 502/1

(57) ABSTRACT

The present invention relates to agglomerated molecular sieves based on a molecular sieve with a pore opening equal to approximately 3 Å, to a process for their preparation and to their use in the drying of organic compounds, particularly alcohols and/or esters, in the gas or liquid phase, by passing the compounds to be dehydrated through a bed of adsorbent based on the said agglomerated sieves.

The present invention relates to a process for the dehydration of organic compounds, particularly alcohols or esters, using agglomerated adsorbents based on molecular sieve with a pore opening equal to approximately 3 Å.

### PRIOR ART

Numerous organic molecules are produced in a medium comprising water or are synthesized by hydrolysis. For example, the alcohols produced by hydration of an olefin, whether ethanol from ethylene or isopropanol from propylene, comprise water once the reaction is complete.

The alcohols obtained by fermentation of materials of agricultural origin, such as beet, maize or sugar cane, also exhibit a high water content.

The esters synthesized from alcohol and carboxylic acid can also be contaminated by traces of water once their synthesis is complete.

In point of fact, numerous uses of organic compounds require that they be devoid of or, at the worst, comprise only minute traces of water, such as applications in the pharmaceutical field; it is necessary to find effective methods for removing as much water as possible.

There exist methods for dehydrating by passing the organic compound to be dehydrated through a bed of adsorbent (desiccant).

For the drying of organic molecules according to these methods, it is known to use, as adsorbents, zeolites with a pore opening of the order of 3 Å where water, unlike the larger organic molecules, can theoretically penetrate. Mention may be made, for example, among zeolites with a pore opening of the order of 3 Å, of zeolites of type A, 28 to 60% (reported in equivalents) of the exchangeable cationic sites of which are occupied by potassium ions, the remainder of the sites being essentially occupied by Na⁺, also referred to as zeolites 3A.

As zeolites exist in the form of very small crystals, typically of less than 10 μm, their use generally requires that they be shaped into objects, such as beads or extrudates, with a larger particle size, typically of between 0.5 mm and 5 mm, this being done in order in particular to avoid pressure drops when these sieves are handled, in particular during operations for charging and discharging adsorption columns. These objects, referred to throughout the continuation as agglomerates, are generally shaped using binders, in particular clay binders, among which may be mentioned sepiolite, attapulgite, montmorillonite or clays of the kaolin family.

FR 2 495 007 or GB 2 088 739 discloses a process for the dehydration of organic solvents by slow passage (surface velocity of the solvent in the column of less than 15 cm/min) through a column comprising a dehydrated molecular sieve based on zeolite 3A where the ratio of the length of the column to the mass transfer region is greater than or equal to 3A4.

U.S. Pat. No. 4,407,662 discloses a process for the adsorption of water of VPSA (Vacuum Swing Adsorption) type comprising a stage of adsorption in the gas phase at the distillation outlet on a column of 3A molecular sieve, followed by a stage of regeneration at a pressure lower than the adsorption pressure with bleeding of a portion of the anhydrous ethanol. The advantage of this adsorption process is that it makes it possible to carry out much faster cycles since the entire process is normally isothermal, except for the heats of adsorption and desorption, which generate a temperature variation which the Applicant Company assesses at less than 14° C., but the adsorption capacity used is much less than the capacity at 3Asaturation.

One of the disadvantages observed during the use of agglomerated conventional commercial 3A molecular sieves in the dehydration of organic compounds, whether in the gas phase or the liquid phase, relates to the formation of undesirable molecules catalysed by the molecular sieve. In the case of the dehydration of ethanol, the formation is observed of acetaldehyde, of ethylene, of diethyl ether, indeed even of more complex molecules, such as paraldehyde, formed by cyclic condensation of acetaldehyde, or acetals and hemiacetals, formed by reaction of ethanol with acetaldehyde3A.

These side reactions also depend on the temperature and thus on the adsorption pressure, which, in the PSA or VPSA processes, is controlled (liquid condensation has to be avoided).

In addition to the amount of water, which has to be very low, certain applications of organic compounds, in particular in the pharmaceutical field, require organic compounds of very high purity where the combined undesirable organic entities do not exceed 10 ppm.

WO 00/34217 discloses a process for drying organic liquids where this liquid to be dehydrated is treated using a 3A molecular sieve which has been subjected beforehand to a treatment intended to reduce its concentration of acid sites to less than 18 3Ammol/g, measured by TPD (Temperature Programmed Desorption) of NH₃. The pretreatment consists in bringing the molecular sieve into contact with a solution of alkali metal salt, preferably of potassium nitrate, followed by several washing operations. By virtue of this pretreatment, carried out on two agglomerated conventional commercial 3A sieves, WO 3A00/34217 shows that success is achieved in reducing, on the one hand, the formation of propylene during the drying of isopropanol and, on the other hand, the formation of diethyl ether during the drying of ethanol. Apart from the fact that this process involves several stages of contact between a solid and a liquid, which complicates it and increases its cost, it is limited in its ability to greatly reduce the content of acid sites in the molecular sieve which are responsible for acid-catalysed reactions, such as intramolecular or intermolecular dehydration or even the formation of acetal. In support of this comment, reference will be made more particularly to the examples which appear in WO 00/34127, where the reduction in the content of acid sites in two commercial zeolites is less than 50%.

### DESCRIPTION OF THE INVENTION

The present invention relates to novel agglomerated molecular sieves based on zeolite 3A which, when they are used in a process for drying liquid or gaseous organic compounds by passing the compound or compounds to be dehydrated over a bed based on the said agglomerated sieves, exhibit the advantage of limiting the formation of undesirable entities obtained by partial conversion of the organic compound or compounds to be dried3A.

The agglomerated sieves according to the invention, with a mean particle size generally of between 1.6 mm and 5 mm, are characterized by

a content of iron, expressed as Fe₂O₃, with respect to the total anhydrous weight of the agglomerate of less than or equal to 0.5% and preferably of less than or equal to 0.3%,

a content of titanium, expressed as TiO₂, with respect to the total anhydrous weight of the agglomerate of less than or equal to 0.2%, preferably of less than or equal to 0.1%.

They can be prepared by agglomeration, according to known techniques, of zeolite powder, for example obtained by hydrothermal synthesis, with an agglomeration binder chosen from clays, such as kaolins, silica and/or alumina. In general, the agglomerated sieves comprise less than 25% of inert binder (inert in the sense of the adsorption) and preferably up to 20% by weight, advantageously up to 10% by weight and more advantageously still in the region of 3A5%.

The binders which are suitable for the present invention will be chosen from conventional agglomeration binders; a person skilled in the art will easily select those having contents of iron and titanium which will make it possible to obtain the agglomerated sieves according to the invention.

The agglomeration can, for example, be carried out by mixing a crystalline zeolite powder (in this instance, 3A or 4A) with water, the binder (generally also in the powder form) and optionally additives for helping with the agglomeration, and then extrusion or pressing of the mixture thus obtained in the form of extrudates or else spraying of this mixture over zeolite agglomerates acting as agglomeration seed. During the spraying, the zeolite agglomerates are subjected to continuous rotation over themselves. This can be carried out by placing the agglomerates in a reactor in rotation about itself around an axis of rotation, the said axis of rotation preferably being inclined with respect to the vertical direction. By this process, commonly denoted in the art by “snowball” process, agglomerates in the form of beads are obtained.3A

The agglomerated sieves thus shaped are subsequently subjected to baking at a temperature of between approximately 400 and 700° C.

An alternative form intended for the production of agglomerated sieves with a low level of inert binder consists in agglomerating the zeolite powder with a binder which can be converted to zeolite, as indicated above, and then converting the binder to zeolite, and then washing and drying the product obtained and activating it at a temperature of between 400 and 700° C.

The binder which can be converted to zeolite can be chosen from clays which can be converted to zeolites, such as kaolin, metakaolin, or halloysite, alone or as a mixture.

The stage of conversion to zeolite consists in converting all or part of the binder which can be converted to zeolite with which the zeolite powder has been agglomerated beforehand by soaking in alkaline solution.

The sieves according to the invention are preferably subjected to a treatment which consists in introducing a basic entity:

either by impregnation in the aqueous phase of the agglomerated and baked 3A sieves using alkali metal hydroxide(s) at ambient temperature (15-30° C.) or of the agglomerated and baked 4A sieves then treated in the aqueous phase using alkali metal hydroxide(s) at a temperature of between 70° C. and the boiling point, s followed by washing operations to remove the excess ionic entities, then by exchange with potassium and by drying,

or by incorporation of hydroxide(s) and/or of carbonate(s) and/or of salt(s) of alkali metal(s) and of organic acid(s), such as acetate, lactate, oxalate, citrate, and the like, during the stage of agglomeration and of shaping. This second alternative form, which is particularly preferred by the Applicant Company, does not require operations of rewetting and of washing the sieve after shaping.

The sieves according to the invention which have been subjected to the latter treatment preferably have a content of alkali metal (preferably sodium and/or potassium), expressed as M₂O, surplus to the exchange capacity of the zeolite (M=preferably Na and/or K), with respect to the total anhydrous weight of the agglomerate of greater than or equal to 0.5% and of less than or equal to 1.4% and preferably of greater than or equal to 0.7% and of less than or equal to 1.1%.

### EXAMPLES

### Description of the Experimental Arrangement

The ability to promote the formation of acetaldehyde during an operation in which ethanol is dried over agglomerated sieves based on zeolite 3A (zeolite of type A, 46% of the CEC (cation exchange capacity) of which is occupied by potassium ions, the remainder being occupied by sodium ions and agglomerated with 20% by weight (with respect to the total weight of the agglomerate) of various binders, the sieves being provided in the form of extrudates with a mean particle size of approximately 1.6 mm, is tested using the arrangement composed of the following elements:

a stock of 96% by weight ethanol (the remaining 4% being water) connected to a peristaltic pump,

a vertical cylindrical reactor (volume=200 ml) placed in an oven fed via the pump; this reactor is charged up to a third of its height with glass beads which are inert with respect to the reactions under consideration and comprises, above, the molecular sieve to be tested, i.e. an amount of the order of 70 ml,

a nitrogen inlet which makes it possible to purge the reactor of the air which is initially present therein,

a condenser at the reactor outlet.

The reactor is charged with sieve and then flushed with nitrogen for 1 hour; the rise in temperature of the oven is programmed so as to reach 140° C. The ethanol is then pumped in the liquid form, which evaporates in the reactor and recondenses at the outlet (temperature=14° C.).

Analyses of the fractions obtained are subsequently carried out by gas chromatography (GC) in order to quantitatively determine the amounts formed.

### Example 1

Several agglomerated sieves are tested for their ability to convert ethanol to acetaldehyde and the contents by weight of minor elements of these sieves and their degree of conversion of ethanol to acetaldehyde are listed in Table 1.

Sieve 1 comprises 20 parts by weight of binder, which comprises calcium, iron, magnesium and titanium in the following proportions: CaO=0.09%; Fe₂O₃=0.77%; MgO=1.15%; TiO₂=0.1%; sieve 2 comprises 20 parts by weight of binder, which comprises calcium, iron, magnesium and titanium in the following proportions: CaO=0.6%; Fe₂O₃=4.7%; MgO=12.5%; TiO₂=0.5%; and sieve 3 comprises 20 parts by weight of binder, which comprises calcium, iron, magnesium and titanium in the following proportions: CaO=0%; Fe₂O₃=1.3%; MgO=0.2%; TiO₂=2%.

Sieve 4 is obtained from sieve 1 by impregnating 50 g of sieve 1 with 40 ml of an aqueous solution comprising 12.64 g of iron nitrate nonahydrate; this amount corresponds to 2.5 g of Fe₂O₃ retained on the solid.

TABLE 1

| | CaO | MgO | Fe₂O₃ | TiO₂ | Conversion |
|---|---|---|---|---|---|
| Agglomerated sieve | (%) | (%) | (%) | (%) | (ppm) |
| 1, according to | 0.018 | 0.23 | 0.15 | 0.018 | 0 |
| the invention | | | | | |
| 2, comparative | 0.12 | 2.5 | 0.94 | 0.1 | 50 |
| 3, comparative | 0 | 0.04 | 0.26 | 0.4 | 7 |
| 4, comparative | 0.017 | 0.21 | 5 | 0.017 | 50 |

### Example 2

Several sieves are tested for their ability to form ethylene and diethyl ether during the dehydration of ethanol and the contents by weight of minor elements of these agglomerated sieves and the results of the catalytic tests are listed in Table 2.

Sieve 5 comprises 20 parts by weight of binder, which comprises calcium, iron, magnesium and titanium in the following proportions: CaO=1%; Fe₂O₃=0.95%; MgO=5.65%; TiO₂=0.05%.

Sieve 6 is obtained by impregnation of 40 g of sieve 5 with 40 ml of an aqueous potassium hydroxide solution comprising 0.8 g of KOH without washing with water. The residual amount of potassium on sieve 6, expressed as amount of K₂O, is 0.8%.

Sieve 7 is obtained by incorporation, during the agglomeration stage, of 0.9 g of K₂CO₃ per 41 g of zeolite powder and 9 g of binder. The residual amount of potassium on sieve 7, expressed as amount of K₂O, is 1.16%.

The amount of acetaldehyde formed on sieves 5 to 7 is also measured; for these three samples, it is of the order of 3 ppm.

TABLE 2

| | CaO | MgO | Fe₂O₃ | TiO₂ | Residual | Conversion, | Conversion, |
|---|---|---|---|---|---|---|---|
| Sieve | (%) | (%) | (%) | (%) | K₂O (%) | ether (ppm) | ethylene (ppm) |
| 5, according | 0.2 | 1.13 | 0.19 | 0.01 | 0 | 15 | 15 |
| to the | | | | | | | |
| invention | | | | | | | |
| 6, according | 0.2 | 1.13 | 0.19 | 0.01 | 0.8 | 2 | 2 |
| to the | | | | | | | |
| invention | | | | | | | |
| 7, according | 0.2 | 1.13 | 0.19 | 0.01 | 1.13 | 3 | <1 |
| to the | | | | | | | |
| invention | | | | | | | |

### Example 3

A sieve is shaped into extrudates with a diameter of 1.6 mm by agglomerating 80% of powder 4A with 20% of binder which can be converted to zeolite comprising calcium, iron, magnesium and titanium in the following proportions: CaO=0.09%; Fe₂O₃=0.77%; MgO=1.15%; TiO₂=0.1%.

After calcining at 550° C. for 2 h, half the sieve is immersed in a sodium hydroxide solution at 100° C. for 2 h (US=4; NaOH=80 g/l), the sieve is then washed with water to pH=11 and exchange with potassium is carried out so as to obtain a degree of exchange equivalent to Example 1 (sieve 8).

A measurement of the H₂O absorption capacity on this sieve under a partial pressure of 0.5 at a temperature of 25° C. shows an improvement of approximately 20% with respect to the sieve which has not been subjected to the treatment in a basic medium at 100° C. (the inert binder represents 4% of the total weight of the final agglomerate).

The catalytic test described in the preceding examples is carried out on both sieves, which have or have not been subjected to the treatment in the sodium hydroxide/potassium hydroxide solution, and the values shown in the table below are found.

TABLE 3

| | Without basic treatment | With basic treatment |
|---|---|---|
| Conversion (ppm) | sieve 1 | sieve 8 |
| Acetaldehyde (%) | 0 | 0 |
| Ether (%) | 7 | 3 |
| Ethylene (%) | 20 | 8 |

An alternative form of this process consists in agglomerating 80% of a 3A powder with 20% of binder which can be converted to zeolite, in baking, in then immersing the solid in a solution of NaOH (80 g/l)+KOH (30 g/l) for 2 h at 100° C., in washing, in order to free the solid from its excess salts, and in drying it at 80° C. The water adsorption capacity, determined under the same conditions as above, is increased by 13% with respect to the sieve which has not been subjected to treatment in the basic medium at 100° C. (the inert binder represents 8% of the total weight of the final agglomerate).

1. Agglomerated molecular sieves based on zeolite 3A and on one or more agglomeration binders, characterized in that their content of iron, expressed as 3AFe2O₃, with respect to the anhydrous weight of sieves is less than or equal to 0.5% and preferably less than or equal to 0.3%, by weight, and their content of titanium, expressed as TiO₂, with respect to the total anhydrous weight of sieves is less than or equal to 0.2%, preferably less than or equal to 0.1%.

2. Sieves according to claim 1, characterized in that their mean particle size is between 1.6 mm and 5 mm.

3. Sieves according to claim 1, characterized in that their content of alkali metal(s), expressed as M₂O, surplus to the exchange capacity of the zeolite (M preferably being sodium and/or potassium), with respect to the total anhydrous weight of sieves is greater than or equal to 0.5% and less than or equal to 1.4% and preferably greater than or equal to 0.7% and less than or equal to 1.1%.

4. Sieves according to claim 1, characterized in that the inert binder is at most 25% of the total weight of the agglomerate, preferably at most 20%, advantageously at most 10% and more advantageously still approximately 5%.

5. Process for the preparation of sieves as defined in claim 1 by agglomeration of zeolite 3A or 4A powder with an agglomeration binder and then baking3A.

6. Process according to claim 4, characterized in that it comprises a treatment consisting in introducing at least one basic entity:
either by impregnation in the aqueous phase of the agglomerated and baked 3A sieves using alkali metal hydroxide(s) at ambient temperature,
or by impregnation in the aqueous phase of the agglomerated and baked 4A sieves using alkali metal hydroxide(s) at a temperature of between 70° C. and the boiling point, followed by washing operations, by optional exchange with potassium and then by drying,
or, preferably, by incorporation of hydroxide(s) and/or of carbonate(s) and/or of salt(s) of alkali metal(s) and of organic acid(s) during the stage of agglomeration of 3A and of shaping.

7. Process for drying organic compounds in the gas phase or in the liquid phase by passing through a bed of adsorbent based on agglomerated sieves as defined in claim 1.

8. Process for drying alcohols and in particular ethanol in the gas phase or in the liquid phase by passing through a bed of adsorbent based on agglomerated sieves as defined in claim 1.

9. Process for drying esters in the gas phase or in the liquid phase by passing through a bed of adsorbent based on agglomerated sieves as defined in claim 1.
